(19) United States
(12) Patent Application Publication
(10) Pub. No.: US 2023/0165870 A1
(43) Pub. Date: Jun. 1, 2023

(54) 6,8-dioxa-3-azabicyclo[3.2.l]Octane Carboxylic Acids And Their Derivatives For Use In The Treatment Of Inflammations

(71) Applicants: MINERVA S.R.L., Firenze (IT); MIMETECH S.R.L., Firenze (IT)

(72) Inventors: Antonio GUARNA, Firenze (IT); Giuseppe CASTRONOVO, Firenze (IT); Michele TANTURLI, Agliana (IT)

(73) Assignees: MINERVA S.R.L., Firenze (IT); MIMETECH S.R.L., Firenze (IT)

(21) Appl. No.: 17/919,248

(22) PCT Filed: Apr. 14, 2021
(86) PCT No.: PCT/IB2021/053062

§ 371 Date: Oct. 14, 2022

(30) Foreign Application Priority Data
Apr. 16, 2020 (IT) 102020000008125

(51) Int. Cl. A61K 31/537; A61P 29/00

(52) U.S. Cl. CPC A61K 31/537; A61P 29/00

(57) ABSTRACT

The present invention describes 3-aza-bicyclo[3.2.1]octane acids of general formula (I), their Salts and Esters, for use as activators of ADAR1 in the treatment of diseases related to acute or chronic inflammations, infective or not., characterized by cytokine storm and/or uncontrolled immune response.

### FIELD OF THE INVENTION

The present invention refers to the field of 3-aza-bicyclo[3.2.1]octane carboxylic acid compounds and their derivatives for use in the treatment of acute or chronic inflammations, infective or not, characterized by cytokine storm and/or uncontrolled immune response.

### STATE OF ART

Inflammation is the immune system’s response to harmful stimuli, such as pathogens (viruses, bacteria, fungi), toxic chemical-biological substances, cell necrosis (myocardial infarction, tissue wounds) and radiation. It represents a defense mechanism that works by removing detrimental stimuli and, at the same time, promoting the healing process. Usually, during inflammatory responses, cellular and molecular events are strictly regulated and aimed at minimizing injury. This mitigation process contributes to the restoration of tissue homeostasis and to a rapid resolution of inflammation, which in this case is defined as acute inflammation. However, when the regulation of the inflammatory process fails, the inflammation becomes uncontrolled and can become chronic leading to several serious inflammatory diseases, or to very dangerous syndromes, such as the so-called “cytokine storm” (Cytokine Storm Syndrome or CSS) [Behrens and Koretzky, arthritis & rheumatology 2017]). Regardless of the etiology, a cascade of biochemical signals necessary for the elimination of the noxious stimulus and healing of damaged tissues is activated during inflammation. In particular, the leukocytes which, in turn, produce inflammatory cytokines, are recalled from the general circulation to the damage sites. Generally, the inflammatory response consists of a series of coordinated events involving both resident tissue cells and those recalled from the blood. Although the type of events triggered during inflammation depends on the nature of the noxious stimulus and by the type of tissue/organ involved, they all share common steps and mechanisms: 1) cell surface receptors recognize the noxious stimuli; 2) activation of the inflammation pathways; 3) release of inflammation markers; 4) recruitment of inflammatory cells; 5) resolution of the inflammation process. This last part is of fundamental importance because it prevents the progression from acute to chronic inflammation stage preventing a prolonged and uncontrolled response that can produce further damages, in addition to those caused by the initial pathogenic stimulus. Obviously, chronic inflammation can occur whenever the initial noxious stimulus is not removed. Normally the inflammation, acute or chronic, is localized, but in some cases, it can become systemic and uncontrolled, giving rise to CSS [Gilroy and De Maeyer, Seminars in Immunology 2015]. CSS is a syndrome characterized by a clinical frame of systemic inflammation, with fever, cytopenia, coagulopathy, multiorgan failure, hyperferritinemia and, if untreated, leads to death. This condition is caused by an abnormal production of cytokines and other inflammatory molecules resulting from an immune system activation out of control. CSS triggers can have several origins: rheumatological, oncological and infective. Among these, the best-known form of CSS is sepsis, a condition caused by a widespread infection, often associated with secondary Haemophagocytic LymphoHistiocytosis (sHLH), a hyper-inflammatory syndrome characterized by hyper-cytokinemia and multiorgan failure. In adults, sHLH is most triggered by viral infections and occurs in 3.7-4.3% of sepsis cases. Key features of sHLH include persistent fever (> 38.5° C.), cytopenia, and hyper-ferritinemia; pulmonary involvement (including Acute Respiratory Distress Syndrome, ARDS) occurs in approximately 50% of patients. However, the consequences of sepsis and CSS are generally not the direct effect of the pathogen (or of another type of initial noxious stimulus) but rather are the result of the uncontrolled immune response to that pathogen. The CSS hallmark is an uncontrolled and dysfunctional immune response involving the continuous activation and expansion of both lymphocytes and macrophages, which secrete large amounts of cytokines causing a cytokine storm. Many clinical features of CSS can be explained by the effects of pro-inflammatory cytokines, such as INterFeron (INF), Tumor Necrosis Factor (TNF), InterLeukins (IL) such as IL-1, IL-6 and IL-18. These pro-inflammatory cytokines are found elevated in most patients with CSS. In this inflammatory context, ADAR1 plays a key role through the modulation of specific proteins involved in the activation of inflammation and in the release of proinflammatory cytokines. For example, during a viral infection, or also in the presence of chemical-physical stress (UV radiation and oxidative stress) or other pathogens, the proteins PKR (Protein Kinase R) and RIG-I (Retinoic acid-Inducible gene I) are activated. These proteins, in their active form, can induce the expression of type 1 interferons (IFN-1) and other pro-inflammatory cytokines. Although IFN-1 is known for its antiviral activity, an excessive production can lead to CSS, for this reason several enzymes capable of regulating its expression to maintain tissue homeostasis exist. One of these is ADAR1 (Adenosine Deaminase Acting on RNA 1), a double-stranded RNA-specific adenosine deaminase capable of binding and modifying viral RNAs and microRNAs (miRNA) [Song C. et al. Genes 2016]. During an infection, ADAR1 binds viral RNA preventing its recognition by the PKR and RIG-I sensors, which are not anymore capable to activate the genes responsible for IFN production. Furthermore, ADAR1, by means of its adenosine deaminase activity, can modify the nucleotide sequence of the viral genome, thus preventing its replication. Finally, a further anti-inflammatory feature of ADAR1 consists in its ability to reduce the expression levels of microRNAs targeting the proteins with anti-inflammatory function (such as, for example, miR-101 and miR-30a). In fact, one of the consequences of the reduction in miR-101 levels is the increase of MKP-1 level, a protein able to turn off p38 MAPK, thus preventing the production of inflammatory mediators responsible for CSS. The pro-inflammatory action of p38 MAPK has been widely documented in several pathologies due to the uncontrolled release of cytokines, including viral ones. Although it plays a central role in the inflammatory response, p38 MAPK represents only one of the many proteins involved in the activation of pro-inflammatory cytokine cascade. To note that the selective p38 MAPK inhibitors are not able to activate ADAR1 and, consequently, such inhibitors only partially allow to counteract the complex mechanisms that characterize CSS, especially when CSS arise from infection.

Furthermore, in viral infection, the activity of ADAR1 consists in modifying the structure of viral RNA by inhibiting its synthesis. Finally, the activation of ADAR1 allows the inactivation of cellular sensors such as PKR and RIG-I avoiding the excessive production of INFs, therefore preventing the uncontrolled release of proinflammatory cytokines.

It is well known that the activation of ADAR1 is one of the innate immunity mechanisms effective in the neutralization of RNA viruses through the editing of their genome [Chung et al., H, Cell 2018].

Despite the existence of various drugs for the treatment of acute and chronic inflammation, currently there is an unmet medical need for the treatment of diseases related to CSS. The treatment of these illnesses mainly consists of immunosuppression complemented by the control of the underlying disease, together with the use of antibiotics or antivirals for patients with an infection [Behrens and Koretzky, arthritis & rheumatology 2017]. As with most inflammatory diseases, CSS can be treated with corticosteroids, or more recently, with therapies aimed at blocking specific cytokines (anti-IL-1, anti-IFN, anti-IL-6 therapies). However, the anti-inflammatory therapies currently available have some limitations because they are exclusively aimed at controlling certain cytokines or because of the resistance to the therapy itself, as in the case of corticosteroids. Nevertheless, the current treatments do not give any trophic support to the tissues and to the organs damaged by uncontrolled inflammation, so even if the inflammation itself can be limited, the systemic damages often persist. These injuries involve, in addition to important organs such as lungs (especially in the case of airway infections), kidneys, liver and heart (in the latter can occur a cardiac failure due to the massive apoptosis) also the vascular endothelium, which must be properly restored. None of the current therapies can limit the multi-organ damage induced by CSS as they are lacking trophic and anti-apoptotic activity.

US2019/0359692 and WO2019/122909 describe p38 MAPK inhibitors for use in the treatment of influenza with severe respiratory tract complications.

Zhou Shangxun et al. (Mediators of Inflammation, 2020; DOI: 10.1155/2020/9607535) demonstrate that ADAR1 alleviates inflammation in a mouse model of sepsis.

WO2004000324, on behalf of the same applicant, describes derivatives of 3-aza-bicyclo [3.2.1] octane, as agonists of human neurotrophins which are therefore valuable for use in the treatment of diseases in which the functions of neurotrophins, in particular the NGF functions, are defective: neurodegenerative disorders of the central nervous system, such as Alzheimer’s Disease (AD), Amyotrophic Lateral Sclerosis (ALS), Huntington’s disease, neuropathies, neural damage caused by hypoxia, ischemia, or trauma , inducing apoptosis of nerve cells; acquired immunodeficiency diseases linked to the reduced bioavailability of NGF, such as age-related immunodeficiency; diseases in which the stimulation of neo angiogenesis is advantageous, such as myocardial infarction, stroke, or peripheral vascular disease; certain eye diseases, such as keratitis of various etiologies, glaucoma, degenerative or inflammatory conditions of the retina. WO2004000324 describes the compound (1S, 4R, 5R, 7S) -3,4-dibenzyl-2-oxo-6,8-dioxo-3-azabicyclo[3.2.1] octan-7-carboxylate of methyl (MT2) as a particularly preferred compound.

WO2013140348, again on behalf of the same Applicant, describes some carboxylic acid derivatives of 3-aza-bicyclo[3.2.1]octane and their medical use, in particular in the treatment of all pathologies related to ischemia-reperfusion, in which the ischemia conditions generated by any reduction or blockage of blood flow, are followed by the subsequent restoration of the oxygen/nutrient supply to the tissue, or for use in medical procedures involving ischemia-reperfusion. WO2013140348 specifically describes acid (1S,4R,5R,7S)-3,4-dibenzil-2-oxo-6,8-dioxane-3-azabicicyclo[3.2.1] octan-7-carboxylic (MT6) and its pharmaceutically acceptable Salts and acid (1S,4R,5R,7S)-3,4-dibenzil-2-oxo-6,8-dioxa-3-azabiciclo[3.2.1]octane-7-carboxylic salt of L-lysine (MT8).

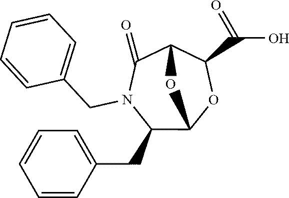 [SMILES: O=C(O)[C@H]1O[C@H]2O[C@@H]1C(=O)N(Cc1ccccc1)[C@@H]2Cc1ccccc1]

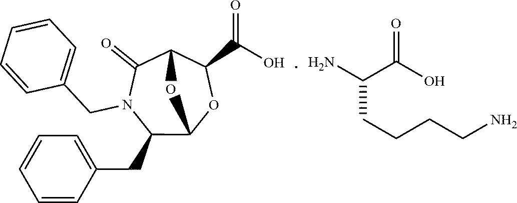

The Applicant has also demonstrated, by means of appropriate preclinical and clinical studies, that MT6 acid, in the form of lysine salt (called MT8), or sodium, or potassium or any other pharmaceutically acceptable salt, dissolved in phosphate or saline buffer, or in any other pharmaceutically acceptable buffer, in the absence or presence of preservatives and excipients, it can be used for the treatment of diseases in which the functions of neurotrophins, in particular the functions of NGF and BDNF, are defective.

On 16-12-2014, MT8 obtained the orphan drug designation from the European Medicines Agency (EMA) for the treatment of neurotrophic keratitis 8EU/3/14/1400. Despite the existence of many drugs used to reduce the damage resulting from acute and chronic inflammation, there is still an unmet medical need for the treatment of these diseases. Therefore, the purpose of the present invention is to provide compounds, at least alternative, for use in the treatment of acute or chronic inflammations in which the so-called CSS cytokine storm syndrome occurs.

A further purpose of the present invention is therefore to provide ADAR1 activating compounds for use in the treatment of severe inflammatory diseases, of infective or non-infective origin, characterized by cytokine storm and/or uncontrolled immune response.

### SUMMARY OF THE INVENTION

The subject-matter of the present invention is a compound of formula (I) for use as an activator of Adenosine Deaminases Acting on RNA 1 (ADAR1) in the treatment of inflammatory diseases, acute or chronic, infective or not, characterized by cytokine storm and/or uncontrolled immune response, said compound of formula (I):

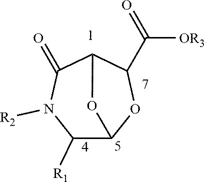

wherein
R₁ is selected from the group consisting of aryl, C₁-₈ alkyl-aryl;
R₂ is selected from the group consisting of C₁-₈ alkyl-aryl;
R₃ is selected from the group H, -C₁-₈ alkyl, C₁-₈ alkyl-aryl;
including pharmaceutically acceptable Salts.

It was unexpectedly discovered through a series of in vitro experiments that the compounds covered by the patent can induce:
i. a high activation of ADAR1 (homodimerization), leading to a significant reduction in the expression of miR-101 and consequently to a decrease in the release of pro-inflammatory cytokines;
ii. reduction of the systemic production of cytokines, upstream of IL-6 in the functional cascade and therefore acting in reduction of the effects deriving from the “cytokine storm” or CSS.

These actions are combined with the activities of:
iii. trophic support to hypoxic tissues at system level through the reduction of damage induced by the ischemia/reperfusion process;
iv. trophic support to the tissues at systemic level through the reduction of the damage induced by the inflammatory process or CSS.

Therefore, the administration of pharmaceutical preparations containing the compounds of formula (I), subject-matter of the patent, as activators of ADAR1, and therefore anti-inflammatory and antiviral agents, are useful for the treatment of diseases related to acute or chronic inflammation characterized by cytokine storm and/or uncontrolled immune response. Furthermore, the administration of pharmaceutical preparations containing the compounds of formula (I), subject-matter of the patent, is preferably, but not exclusively, useful for the treatment of respiratory tract diseases, and in particular, but not exclusively, induced by viral factors, such as Severe Acute Respiratory Syndrome (SARS) caused by coronavirus or other viruses, limiting biochemical and functional damage in the severely damaged lung endothelium, as well as in hypoxic tissues of different organs (brain, kidney, liver, etc.), often already compromised by pre-existing or concomitant pathologies.

### DETAILED DESCRIPTION OF THE INVENTION

In the present invention, unless otherwise specified, the terms alkyl, aryl, alkylaryl, are to be understood as follows:
C₁₋₈ alkyl (Alk₁₋₈), refers to linear or branched alkyl radicals, having single bonds, C—C. Examples of alkyl groups according to the present invention include, but without limitation, methyl, ethyl, propyl, isopropyl, butyl, pentyl, slender, heptile, octyl.
the term “aryl” indicates a group containing one or more unsaturated rings, each ring having from 5 to 8 members, preferably 5 or 6 members. Examples of aryl groups include, but are not limited to phenyl, biphenyl and naphthyl.

According to the present invention, the aryl groups can be replaced with one or more groups, and preferably one or two groups selected from the group consisting of halogen, cyano, nitro, amino, hydroxy, carboxylic acid, carbonyl, and C₁-₆ alkyl (Alk₁₋ ₆). The term “halogen” refers to fluorine, chlorine, bromine, and iodine.

In the compounds of the present invention preferably R1 is CH₂Ph.

Preferably, R2 is CH₂Ph.

Preferably, R3 is H or CH₃.

Optionally, the phenyl groups can be replaced with one or more groups, and preferably one or two groups selected from the group consisting of X, CN, NO₂, NH₂, OH, COOH, (C═O)Alk₁₋₆; where X is chosen from the group consisting of F, Cl, Br and I.

Among the compounds of formula (I) are preferred those wherein:
R1 is CH₂Ph; and
R2 is CH₂Ph; and
R3 is H or CH₃; and
where the phenyl groups can optionally be substituted with one or more groups, and preferably one or two groups selected from the group consisting of X, CN, NO₂, NH₂, OH, COOH, (C═O) Alk₁₋₆; where X is chosen from the group consisting of F, Cl, Br and I.

For the purposes of the present invention, compounds of formula (IA) and (IB) are more preferred:

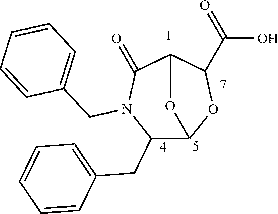

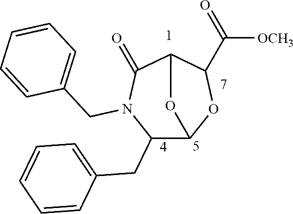

Such compounds can clearly occur in various stereochemical configurations

| Compound | Stereocentre | | | |
|---|---|---|---|---|
| | 1 | 4 | 5 | 7 |
| 1A | R | R | S | S |
| 2A | S | S | R | R |
| 3A | R | R | R | R |
| 4A | S | S | S | S |
| 5A | S | R | R | R |
| 6A | R | S | S | S |
| 7A | R | S | R | R |
| 8A | S | R | S | S |
| 9A | R | R | S | R |
| 10A | S | S | R | S |
| 11A | R | R | R | S |
| 12A | S | S | S | R |
| 13A | R | S | R | S |
| 14A | S | R | S | R |
| 15A | R | S | S | R |
| 16A (MT6) | S | R | R | S |

| Compound | Stereocentre | | | |
|---|---|---|---|---|
| | 1 | 4 | 5 | 7 |
| 1B | R | R | S | S |
| 2B | S | S | R | R |
| 3B | R | R | R | R |
| 4B | S | S | S | S |
| 5B | S | R | R | R |
| 6B | R | S | S | S |
| 7B | R | S | R | R |
| 8B | S | R | S | S |
| 9B | R | R | S | R |
| 10B | S | S | R | S |
| 11B | R | R | R | S |
| 12B | S | S | S | R |
| 13B | R | S | R | S |
| 14B | S | R | S | R |
| 15B | R | S | S | R |
| 16B (MT2) | S | R | R | S |

For the purposes of the present invention, the compounds (1S, 4R, 5R, 7S) -3,4-dibenzyl-2-oxo-6,8-dioxo-3-azabicyclo[3.2.1] octane-7-carboxylate of methyl (MT2), the (1S, 4R, 5R, 7S) -3,4-dibenzyl-2-oxo-6,8-dioxo-3-azabicyclo [3.2.1] octane-7-carboxylic acid named (MT6) and its pharmaceutically acceptable Salts, among the MT6 Salts, the following Salts are of particular interest: potassium Salts, sodium Salts, lysine Salts, organic and inorganic quaternary ammonium Salts. A particularly preferred compound is therefore the (1S, 4R, 5R, 7S) -3,4-dibenzyl-2-oxo-6,8-dioxa-3-azabicyclo[3.2.1]octane-7-carboxylic acid L-lysine Salt (MT8).

It has been observed that the compounds of formula (I), described above, are able to reduce inflammatory cytokines produced by LPS-activated human monocytes/macrophages and by LPS-activated human dendritic cells (see FIG. 1). In particular, in pro-inflammatory conditions, a compound of the invention is capable of activating ADAR1, an enzyme which, through its RNA-editing activity, is able to reduce the expression of miR-101, a microRNA (miRNA) involved in the proinflammatory response. The activation of ADAR1 by a compound of the invention was observed in HEK-293 TrkA cell lines (see FIG. 2). In fact, it has been shown that the administration of the compound induces a marked increase in the homodimeric form ADAR1/ADAR1, compared to untreated cells, promoting the formation of the active form of the protein, and thus favoring the editing process. The ability of ADAR1 to reduce the expression of miR-101 was observed in cells treated with the compound of the invention in the presence or absence of ADAR1 knock-down (FIG. 3 and FIG. 4).

A higher activity of ADAR1, at the level of each individual cell, may prove further beneficial in protecting cells from damage induced by any viral infection and inflammatory processes, not necessarily of an infective nature.

The activity of ADAR1 is in fact dual:
in a non-infective inflammatory process, ADAR1 is capable to bind and edit miRNAs, which, once modified, cannot recognize their target sequences and are therefore degraded; in particular, this occurs for miR-101 but also for miR-30a, a miRNA whose expression determines the increase of pro-inflammatory cytokines such as TNF-α and IL-6.
moreover, in a viral infection, the activity of ADAR1 consists in the structural modification of the viral RNA by inhibiting its synthesis.

Finally, the activation of ADAR1 allows the deactivation of cellular sensors such as PKR and RIG-I avoiding the excessive production of INF, thus preventing uncontrolled release of pro-inflammatory cytokines.

In conclusion, a compound of the invention has a powerful anti-inflammatory and antiviral activity as it can determine:
i) a reduction in viral load and consequent infection through the increase in cytoplasmic levels of ADAR1, an enzyme capable of damaging the genome of RNA viruses;
ii) a reduction in the systemic production of cytokines and consequent reduction of the effects deriving from the so-called “cytokine storm” in the patient, through the activation of ADAR1 and the consequent decrease of miR-101;
iii) a reduction in the damage induced by the ischemia/reperfusion process that occurs in severe inflammatory states, through metabolic support to hypoxic tissues; Hence, the compounds for use according to the present invention, as activators of ADAR1, are effective anti-inflammatory and antiviral agents, and are therefore useful for the treatment of diseases related to acute or chronic inflammation characterized by cytokine storm and/or uncontrolled immune response.

Specifically, the compounds for use according to the present invention, as activators of ADAR1, are potentially useful for the treatment of infective diseases of viral origin, such as:
Herpes Virus,
Epstein-Barr virus,
Cytomegalovirus,
Adenovirus,
HPV,
Coronavirus,
Enterovirus,
Rotavirus,
Parvovirus,
Influenza A virus,
Ebolavirus,
members of the genus Marburgvirus,
members of the dengue virus species,
hepatitis A (HAV), B (HBV), C (HCV) infections,
Pan-encephalitis (SSPE) in the measles virus infections,
Haemorrhagic fever virus (Arenaviridae, Bunyaviridae, Filoviridae, Falviviridae, and Togaviridae),
Measles virus,
Mumps virus,
Rubella virus,
Parechovirus,
Human T-lymphotropic virus.

The compounds for use according to the present invention, as activators of ADAR1 and therefore anti-inflammatory agents, are also potentially useful for the treatment of infective diseases characterized by cytokine storm,
i) of bacterial origin such as:
   Aeromonas hydrophila,
   Brucella sp.,
   Chlamydia sp.,
   Clostridium sp.,
   Escherichia coli,
   Legionella sp.,
   Mycobacteria,
   Salmonella,
   Staphylococcus aureus,
   Acinetobacter baumannii;
   Mycobacterium tuberculosis
   Mycoplasma pneumoniae
ii) of origin from parasites and fungi such as:
   Plasmodium sp.,
   Leishmania sp.
   Toxoplasma gondii,
   Entamoeba histolytica,
   Babesia sp.,
   Ascaris lumbricoides,
   Helminths,
   Candida albicans,
   Histoplasma,
   Cryptococcus neoformans,
   Pneumocystis sp.,
   Penicillium marneffei.
iii) of origin from zoonoses such as:
   Brucella,
   Rickettsiae,
   Ehrlichia,
   Coxiella burnetiid,
   Mycobacterium avium,
   Clostridium,
   Leptospira.

The compounds for use according to the present invention, as activators of ADAR1, are also potentially useful for the treatment of diseases of non-infective origin causing a decrease in the state of acute and chronic inflammation such as:
sepsis,
Hemophagocytic lymphohistiocytosis,
Still’s disease adult onset (AOSD),
Chronic liver inflammation,
obesity,
atherosclerosis,
periodontitis,
cirrhosis,

The compounds for use according to the present invention, as activators of ADAR1 and therefore anti-inflammatory agents, are therefore also potentially useful for the treatment of autoimmune and degenerative diseases such as:
Hemophagocytic lymphohistiocytosis,
Lymphoproliferative syndromes,
Primary and acquired immunodeficiencies not due to NGF deficiency,
hereditary symmetric dyschromatosis (DSH),
Aicardi-Goutieres syndrome (AGS),
rare genetic diseases linked to IL-⅟inflammasome disorders,
IFN-mediated disorders,
NF-κB/ubiquitinin mediated disorders
Muckle-Wells syndrome,
hyper-IgD syndrome,
pediatric granulomatous arthritis,
ADA2 deficiency,
sepsis,
Arthritis/Osteoarthritis,
Juvenile idiopathic arthritis,
Lupus erythematosus,
Kawasaki disease,

The compounds for use according to the present invention, as activators of ADAR1 and therefore anti-inflammatory and antiviral agents, are particularly useful also for the treatment of inflammatory diseases of the respiratory tract such as:
Severe Acute Respiratory Syndrome (SARS) induced by coronavirus or other viruses,
asthma,
chronic obstructive pulmonary disease (COPD),
bronchiectasis,
pulmonary interstitial disease or pulmonary disease,
bronchiolitis,
bronchopulmonary dysplasia (BPD) of the premature infant,
tuberculosis,
whooping cough;
acute inhalation injuries due to exposure to noxious and toxic substances,
occupational respiratory tract infections such as
   Legionellosis,
   Q fever,
pulmonary interstitial diseases induced by professional activities such as
   pneumoconiosis,
   lung diseases from exposure to metals,
   extrinsic allergic alveolitis,
   Ardystil syndrome;
rare lung diseases such as:
   pulmonary vasculitis,
   idiopathic eosinophilic pneumonia,
   pulmonary alveolar proteinosis,
   lymphangioleiomyomatosis (LAM),
   pulmonary Langerhans cell histiocytosis,
   Birt-Hogg-Dube syndrome.

The compounds for use according to the present invention can be formulated in conventional pharmaceutical compositions, which may include one or more pharmaceutically acceptable excipients and/or diluents.

The administration of these compositions can be carried out by any conventional route of administration, for example parenterally in the form of injectable solutions or suspensions, orally, topically, nasally, subcutaneously, subconjunctival, etc. The above compositions can be in the form of tablets, capsules, solutions, dispersions, suspensions, liposomal formulations, microspheres, nanospheres, foams, creams and ointments, emulsions, microemulsions and nanoemulsions, and aerosols, and can also be prepared in such a way as to make a controlled or delayed release of the active ingredient.

All the above-described pharmaceutical compositions can comprise at least one of the present compounds of formula (I) as active ingredient, optionally in combination with other active ingredients or adjuvants, selected according to the pathological conditions to be treated.

The present invention can be better understood in the light of the following embodiment examples.

### BRIEF DESCRIPTION OF THE FIGURES

FIG. 1 - The graphs show the production of IL-1β, TNF-α and IL-6 in human monocytes and human dendritic cells stimulated with LPS, in the presence or absence of the MT8 compound. It is evident that the production levels of proinflammatory cytokines are significantly lower in both monocytes and dendritic cells treated with the MT8 compound compared to untreated cells.

FIG. 2 - Effect of MT8 on the activation of the homodimeric complex (active form of the protein) of ADAR1. The gel shows the induction of the ADAR1/ADAR1 homodimeric complex in HEK-293 TrkA cells treated with the MT8 compound. The graph shows the quantitative determination, performed by densitometry, of the gel bands and it is expressed as the ratio between the band density of the homodimeric complex ADAR1/ADAR1 and the monomer ADAR1. The ADAR1/ADAR1 homodimer levels induced by the MT8 compound are significantly higher than in the control.

FIG. 3 - Effect of MT8 on miR-101 reduction in an in vitro model of inflammation. The graph shows the ability of MT8 to decrease the expression of intracellular miR-101. This event was observed on human monocytes and dendritic cells cultured in the presence of LPS, one of the compounds with the highest pro-inflammatory activity. The level of miR-101 was quantified by Real-time PCR using 5 s ribosomal RNA for signal normalization and relative increase calculated applying the 2^{-ΔCt} method.

FIG. 4 - Effect of MT8 on miR-101 expression levels following ADAR1 knock-down. The graph shows the ability of the MT8 compound to decrease the expression of miR-101 in HEK-293 TrkA cells transfected with scrambled (control) siRNA but not on cells transfected with specific siRNA for ADAR1. The level of miR-101 was quantified by Real-time PCR using 5 s ribosomal RNA for signal normalization and relative increase calculated applying the 2^{-ΔCt} method.

### EXPERIMENTAL PART

### Materials

Acid (1S, 4R, 5R, 7S)-3,4-dibenzyl-2-oxo-6,8-dioxa-3-azabicyclo[3.2.1]octane-7-carboxylic L-lysine Salt (MT8) was prepared as described in WO2013140348.

### Experiment 1 - Effect of MT8 on the Production of IL-1β, TNF-α and IL-6 in Human Monocytes and Human Dendritic Cells Stimulated With LPS

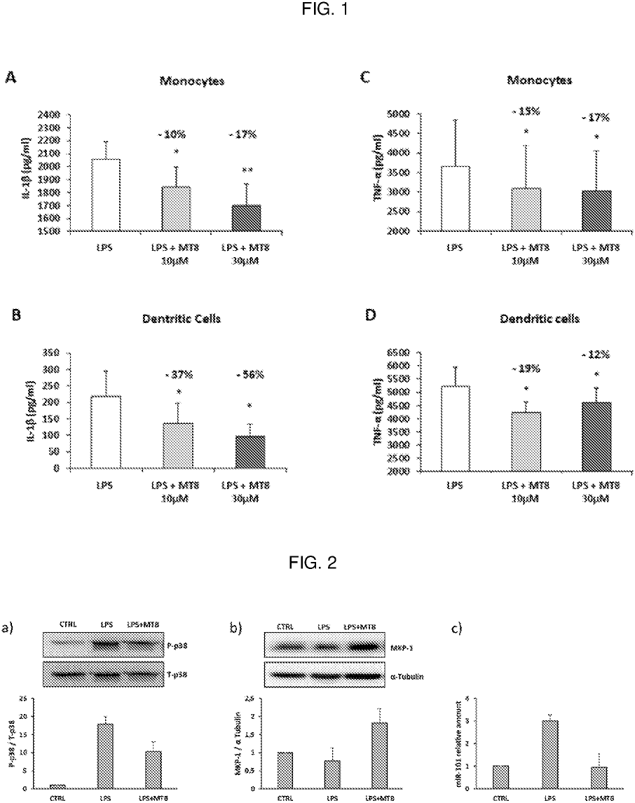

LipoPoliSaccharide (LPS) is an endotoxin that induces a strong immune reaction. In the presence of LPS, immune system cells such as monocytes and dendritic cells react by producing high amounts of inflammatory cytokines such as IL-1β, TNFα and IL-6. To test the ability of MT8 to modulate the production of these cytokines, human monocytes (isolated from buffy coat using anti-CD14 antibody) and human monocytes-derived dendritic cells (MDCs) were cultured at 10⁶ cells/ml in complete medium and stimulated with 50 ng/ml of LPS, in the presence or absence of MT8 at a concentration of 10 or 30 µM. After 18 hours of incubation, the supernatants of both cell types were collected and the production of IL-1β, TNF-α, and IL-6 was evaluated by Luminex multiplex assay technology. The results obtained (FIG. 1) showed that in the experimental conditions described above, LPS was able, as expected, to induce the production of IL-1β, TNF-α and IL-6 and that such production is reduced by the treatment with MT8. In particular, MT8 was able to decrease the amount of IL-1β in a dose-dependent manner in both monocytes and dendritic cells. In the latter, the reduction recorded was over 50% (FIG. 1B), while in monocytes the reduction was about 17% (FIG. 1A). In any case, this reduction is statistically significant. Similar results were obtained regarding the production of TNF-α which, in the presence of MT8, is reduced by about 17% in monocytes (FIG. 1C) and by 19% in dendritic cells (FIG. 1D). Again, the reduction observed in the presence of MT8 is statistically significant. Finally, the production of IL-6 is also significantly reduced in the presence of MT8, by about 70% in monocytes (FIG. 1E) and by about 65% in dendritic cells (FIG. 1F). Also, in the latter case the reduction is statistically significant.

In summary, the experiments show that in all cases the production levels of the three cytokines are significantly lower in both monocytes and dendritic cells treated with the MT8 compound compared to untreated cells.

### Experiment 2 - Effect of MT8 on the Activation of the Homodimeric Complex of ADAR1

In order to study the effect of the MT8 compound on the ADAR1 enzyme, HEK-293 TrkA cells were cultured in serum-free medium for 18 hours and incubated with or without 10 µM of MT8 for another 60 minutes. The cells were then lysed in RIPA buffer (50 mM Tris-HCI, pH 7.4; 150 mM NaCl; 2 mM EDTA; 1 mM NaF; 1 mM sodium orthovanadate, 1% NP-40) and the proteins immunoprecipitated with Anti-ADAR1 antibody and subjected to biochemical analysis by Western Blot. Briefly, 500 µg of total protein was immunoprecipitated using a specific Anti-ADAR1 antibody. The immunoprecipitated product was loaded onto polyacrylamide gel and transferred onto the PVDF membrane. The membrane was then incubated with specific anti-ADAR1 antibodies for signal detection. The analysis allowed to highlight the presence of homodimeric complexes ADAR1/ADAR1 and of the monomeric forms of ADAR1 p150 and p110. The quantitative determination of the homodimer complex of ADAR1, performed by densitometry, was expressed as the ratio between the density of the band of the homodimer ADAR1/ADAR1 and that of the monomer ADAR1 p110. The data obtained showed (FIG. 2) that the administration of the MT8 compound induces a high increase in the homodimeric (active) form of ADAR1 compared to untreated cells, thus promoting the editing process.

### Experiment 3 - Effect of MT8 on the Expression of miR-101 in an in Vitro Model of Inflammation

The production of IL-1β and TNFα and IL-6, triggered by pro-inflammatory stimuli such as LPS, is determined by the activation of specific pathways, among which the expression of miR-101 is involved.

In order to study the effect of MT8 on the activity of miR-101 in the pro-inflammatory setting, human monocytes isolated from buffy coat were stimulated with 1 µg/ml of LPS in the presence or absence of MT8 at the final concentration of 1 0 µM. After 60 minutes, the cells were lysed in TRIzol for the extraction of total RNA, which was then used for the quantification of miR-101 by Real-time PCR. The results obtained by Real-time PCR showed that the administration of the MT8 compound induced a strong decrease in the miR-101 expression level compared to monocytes treated with LPS alone. The quantitative determination was performed using the 5 s ribosomal RNA gene as housekeeping and the relative increase calculated applying the 2^{-ΔCt} method.

The graph shows the ability of the MT8 compound to decrease the expression of miR-101 within the cell. This event was observed on human monocytes cultured in the presence of LPS, one of the compounds with the greatest pro-inflammatory activity.

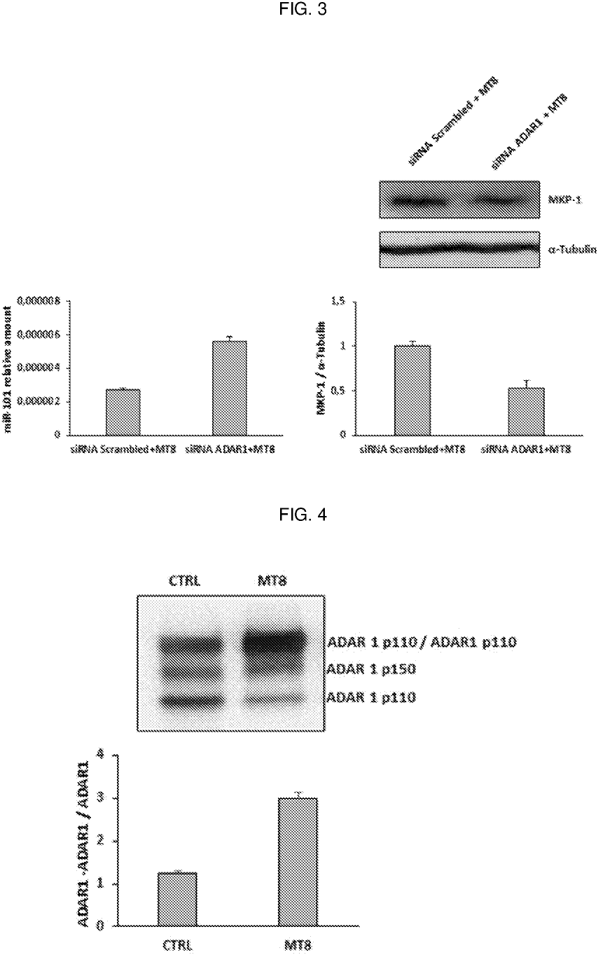

It was therefore surprisingly discovered that in cellular and tissue systems the exposure to MT8, one of the compounds object of the patent, determines, within 1 hour, the decrease of miR-101, this explains the rapid decrease of pro-inflammatory cytokines immediately after treatment with MT8. In summary, the data obtained (FIG. 3) show that in pro-inflammatory conditions, the compound MT8, by decreasing the expression of miR-101, is able to determine a decrease of the cellular inflammation state.

### Experiment 4 - Effect of MT8 on miR-101 Expression Levels Following ADAR1 Knock-Down

In order to study the effect of MT8 on miR-101 regulatory mechanisms under metabolic stress conditions, HEK-293 TrkA cells were transfected with ADAR1-specific siRNA or control siRNA (scrambled) at the final concentration of 50 nM. After 48 hours, the cells were incubated in serum-free medium for 18 hours and stimulated with MT8 at a concentration of 10 µM for a further 60 minutes. The cells were lysed with TRIzol for the extraction of total RNA, used for the assay of miR-101 by Real Time PCR. The quantitative determination of miR-101 was performed using the 5 s ribosomal RNA gene as the housekeeping gene and the relative increase calculated applying the 2^{-ΔCt} method. The results obtained demonstrated that administration of the MT8 compound induced a marked decrease in miR-101 expression level in scrambled siRNA-transfected cells compared to ADAR1-specific siRNA-transfected cells, indicating that the decrease in miR-101 levels is regulated by ADAR1 activity.

Taken together, these data show (FIG. 4) that in the experimental conditions described above, treatment with MT8 is capable of decreasing levels of miR-101 through the activation of ADAR1, thus allowing the production of pro-inflammatory cytokines to be blocked.

1. A method of treating inflammatory diseases characterized by cytokine storm and/or uncontrolled immune response, said method comprising administering to a subject in need thereof a compound of formula (I) as an activator of Adenosine Deaminase Acting on RNA 1 (ADAR1), said compound of formula (I) 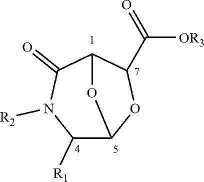
wherein
R₁ is selected from the group consisting of aryl, C₁₋₈alkyl-aryl;
R₂ is selected from the group consisting of C₁₋₈alkyl-aryl;
R₃ is selected from the group H, -C₁₋₈ alkyl, C₁₋₈ alkyl-aryl; and
including pharmaceutically acceptable salts.

2. The method according to claim 1 wherein
R₁ is CH₂Ph; or
R₂ is CH₂Ph; or
R₃ is H or CH₃; and
optionally the phenyl groups can be substituted with one or more groupings, and preferably one or two groupings selected from the group consisting of X, CN, NO₂, NH₂, OH, COOH, (C═O) Alk₁₋₆; where X is chosen from the group consisting of F, Cl, Br and I.

3. The method according to claim 2 wherein
R₁ is CH₂Ph; and
R₂ is CH₂Ph; and
R₃ is H or CH₃; and
where the phenyl groups can optionally be substituted with one or more groupings, and preferably one or two groupings selected from the group consisting of X, CN, NO₂, NH₂, OH, COOH, (C═O) Alk₁₋₆; where X is chosen from the group consisting of F, Cl, Br and I.

4. The method according to claim 3 wherein said compound is of formula (IA) or (IB) 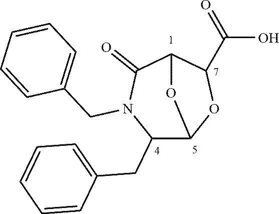 [SMILES: O=C(O)C1OC2OC1C(=O)N(Cc1ccccc1)C2Cc1ccccc1] 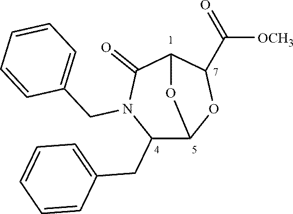 [SMILES: COC(=O)C1OC2OC1C(=O)N(Cc1ccccc1)C2Cc1ccccc1]
.

5. The method according to claim 4 wherein the compound is selected from the group consisting of (1S, 4R, 5R, 7S) -3,4-dibenzyl-2-oxo-6,8-dioxa-3-azabicyclo[3.2.1]methyl octane-7-carboxylate (MT2), the acid (1S, 4R, 5R, 7S) -3,4-dibenzyl-2-oxo-6,8-dioxa-3-azabicyclo [3.2.1] octane -7-carboxylic called (MT6) and its pharmaceutically acceptable salts.

6. The method according to claim 5 wherein the compound, is the acid (1S, 4R, 5R, 7S) -3,4-dibenzyl-2-oxo-6,8-dioxa-3-azabicyclo[3.2.1] octane-7-carboxylic L-lysine Salt.

7. The method according to claim 1 wherein said inflammatory diseases are acute or chronic, and origin from infection.

8. The method according to claim 7 wherein said acute or chronic inflammatory diseases origin from infection induced by a viral factor.

9. The method according to claim 8 wherein the viral factor is selected from the group consisting of:
Herpes Virus,
Epstein-Barr virus,
Cytomegalovirus,
Adenovirus,
HPV,
Coronavirus,
Enterovirus,
Rotavirus,
Parvovirus,
Influenza A virus,
Ebolavirus,
members of the genus Marburgvirus,
members of the dengue virus species,
hepatitis A (HAV), B (HBV), C (HCV) virus infections,
Pan-encephalitis (SSPE) in measles virus,
Hemorrhagic fever virus (Arenaviridae, Bunyaviridae, Filoviridae, Falviviridae, and Togaviridae),
Measles virus,
Mumps virus,
Rubella virus,
Parechovirus,
Human T-lymphotropic virus, and
Influenza and parainfluenza viruses.

10. The method according to claim 7 wherein the inflammatory diseases are serious:
i) of bacterial origin selected from the group consisting of
Aeromonas hydrophila,
Brucella sp.,
Chlamydia sp,
Clostridium sp.,
Escherichia coli,
Legionella sp.,
Mycobacteria, Mycobacterium tuberculosis,
Salmonella,
Staphylococcus aureus, and
Acinetobacter baumannii;
ii) of origin from parasites and fungi selected from the group consisting of:
Plasmodium sp.,
Leishmania sp.,
Toxoplasma gondii,
Entamoeba histolytica,
Babesia sp.,y
Ascaris lumbricoides,
Helminths,
Candida albicans,
Histoplasma,
Cryptococcus neoformans,
Pneumocystis sp., and
Penicillium marneffei;
iii) of origin from zoonoses selected from the group consisting of:
Brucella,
Rickettsiae,
Ehrlichia,
Coxiella burnetiid,
Mycobacterium avium,
Clostridium, and
Leptospira.

11. The method according to claim 1 wherein the inflammatory diseases are severe and of autoimmune and/or degenerative origin selected from the group consisting of:
Hemophagocytic lymphohistiocytosis,
Lymphoproliferative syndromes,
Primary and acquired immunodeficiencies not due to NGF deficiency,
hereditary symmetric dyschromatosis (DSH),
rare genetic diseases linked to IL-1 / inflammasome disorders,
IFN-mediated disorders,
NF-κB / ubiquitinin mediated disorders,
Muckle - Wells syndrome,
hyper-IgD syndrome,
pediatric granulomatous arthritis,
ADA2 deficiency,
sepsis,
Arthritis / Osteoarthritis,
Juvenile idiopathic arthritis,
Lupus erythematosus,
Kawasaki disease.

12. The method according to claim 1 wherein the inflammatory diseases are severe inflammatory diseases of the respiratory tract selected from the group consisting of:
Severe Acute Respiratory Syndrome (SARS) induced by coronavirus or other viruses,
asthma,
chronic obstructive pulmonary disease (COPD),
bronchiectasis,
pulmonary interstitial disease or pulmonary disease,
bronchiolitis,
bronchopulmonary dysplasia (BPD) of the premature infant,
tuberculosis,
whooping cough,
acute inhalation injuries due to exposure to noxious and toxic substances,
occupational respiratory tract infections selected from the group consisting of
Legionellosis,
Q fever;
interstitial lung diseases induced by professional activities selected in the group consisting of:
pneumoconiosis,
lung diseases from exposure to metals,
extrinsic allergic alveolitis,
Ardystil syndrome;
rare lung diseases selected from the group consisting of:
pulmonary vasculitis,
idiopathic eosinophilic pneumonia,
pulmonary alveolar proteinosis,
lymphangioleiomyomatosis (LAM),
pulmonary Langerhans cell histiocytosis,
Birt-Hogg-Dubé syndrome.
Hemophagocytic lymphohistiocytosis.

13. The method according to claim 1 wherein the compound of formula (I) is administered in combination with at least one other active ingredient or adjuvant, chosen according to the pathological conditions to be treated.
